(19) United States
(12) Patent Application Publication
(10) Pub. No.: US 2012/0309744 A1
(43) Pub. Date: Dec. 6, 2012

(54) DOSING REGIMENS AND METHODS FOR TREATING OR PREVENTING HEPATOCELLULAR CARCINOMA

(75) Inventors: John NEUSTADT, Bozeman, MT (US); Steve PIECZENIK, Miami Beach, FL (US)

(73) Assignee: NBI Pharmaceuticals, Inc., Bozeman, MT (US)

(21) Appl. No.: 13/151,160

(22) Filed: Jun. 1, 2011

(51) Int. Cl. A61K 31/122; A61K 31/403; A61P 35/00; A61K 31/55; A61K 31/4166; A61K 31/40; A61K 31/47

(52) U.S. Cl. 514/213.01; 514/682; 514/423; 514/412; 514/307; 514/392

(57) ABSTRACT

The invention encompasses dosing regimens in which a subject is administered menatetrenone over a period of time to establish initial therapeutic baseline blood concentration of the menatetrenone followed by a maintenance therapy to maintain therapeutic blood concentrations. In other embodiments, the invention encompasses methods of treating hepatocellular adenocarcinoma in a subject in need thereof comprising administering to a subject a therapeutically effective dosing regimen of menatetrenone.

### I. FIELD OF THE INVENTION

The invention encompasses dosing regimens in which a subject is administered menatetrenone over a period of time to establish initial therapeutic baseline blood concentration of the menatetrenone followed by a maintenance therapy to maintain therapeutic blood concentrations. In other embodiments, the invention encompasses methods of treating hepatocellular adenocarcinoma in a subject in need thereof comprising administering to a subject a therapeutically effective dosing regimen of menatetrenone.

### II. BACKGROUND OF THE INVENTION

Hepatocellular carcinoma (HCC) is one of the most common malignant tumors worldwide and is closely associated with chronic liver disease. Approximately 80-90% of cases occur in patients with liver cirrhosis, which is believed to be the most important risk factor for HCC. Despite the progression of therapeutic approaches, including radiofrequency ablation (RFA), the long-term prognosis for patients with HCC is still poor because of the high relapse rate and the frequent incidence of intrahepatic metastases.

HCC is the most common type of liver cancer. Most cases of HCC are secondary to either a viral hepatitide infection (hepatitis B or C) or cirrhosis (alcoholism being the most common cause of hepatic cirrhosis). Compared to other cancers, HCC is quite a rare tumor in the United States, In countries where hepatitis is not endemic, most malignant cancers in the liver are not primary HCC but metastasis of cancer from elsewhere in the body (e.g., the colon). Treatment options of HCC and prognosis are dependent on many factors but especially on tumor size and staging. Tumor grade is also important. High-grade tumors will have a poor prognosis, while low-grade tumors may go unnoticed for many years, as is the case in many other organs, such as the breast, where a ductal carcinoma in situ (or a lobular carcinoma in situ) may be present without any clinical signs and without correlate on routine imaging tests, although in some occasions it may be detected on more specialized imaging studies like MR mammography.

Hepatocellular carcinoma, like any other cancer, develops when there is a mutation to the cellular machinery that causes the cell to replicate at a higher rate and/or results in the cell avoiding apoptosis. In particular, chronic infections of hepatitis B and/or C can aid the development of hepatocellular carcinoma by repeatedly causing the body's own immune system to attack the liver cells, some of which are infected by the virus, others merely bystanders. While this constant cycle of damage followed by repair can lead to mistakes during repair which in turn lead to carcinogenesis, this hypothesis is more applicable, at present, to hepatitis C. Chronic hepatitis C causes HCC through the stage of cirrhosis. In chronic hepatitis B, however, the integration of the viral genome into infected cells can directly induce a non-cirrhotic liver to develop HCC. Alternatively, repeated consumption of large amounts of ethanol can have a similar effect. Besides, cirrhosis is commonly caused by alcoholism, chronic hepatitis B and chronic hepatitis C. The toxin aflatoxin from certain Aspergillus species of fungus is a carcinogen and aids carcinogenesis of hepatocellular cancer by building up in the liver. The combined high prevalence of rates of aflatoxin and hepatitis B in settings like China and West Africa has led to relatively high rates of heptatocellular carcinoma in these regions. Other viral hepatitides such as hepatitis A have no potential to become a chronic infection and thus are not related to hepatocellular carcinoma.

The inventors have surprisingly found that dosing regimens of menatetrenone resulted in increased therapeutic efficacy in subjects suffering from hepatocellular carcinoma.

### III. SUMMARY OF THE INVENTION

The invention generally encompasses methods of treating hepatocellular carcinoma comprising administering a therapeutically effective amount of menatetrenone to a subject in need thereof.

In one embodiment, the invention encompasses methods of inhibiting vascular endothelial growth factor comprising administering menatetrenone to a subject according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain illustrative embodiments, the subject is suffering from hepatocellular adenocarcinoma.

In certain illustrative embodiments, the amount of menatetrenone administered in the maintenance phase is less than the amount of menatetrenone administered in the initial induction phase.

In certain illustrative embodiments, the initial induction phase period is about 30 to 120 days.

In certain illustrative embodiments, the initial induction phase period is about 60 to 90 days.

In certain illustrative embodiments, the maintenance phase period is at least 60 days.

In certain illustrative embodiments, the maintenance phase period is at least 90 days.

In certain illustrative embodiments, the maintenance phase period is at least 120 days.

In certain illustrative embodiments, the menatetrenone is administered intravenously, orally, or subcutaneously.

In another embodiment, the invention further comprises administering an angiotensin converting enzyme inhibitor.

In certain illustrative embodiments, the angiotensin converting enzyme inhibitor is enalapril, ramipril, quinapril, perindopril, lisinopril, benazepril, imidapril, or zofenopril.

In certain illustrative embodiments, the angiotensin converting enzyme inhibitor is ramipril.

In another embodiment, the invention encompasses methods of treating hepatocellular adenocarcinoma comprising administering to a subject in need thereof a therapeutically effective amount of menatetrenone according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain illustrative embodiments, the invention further comprises administering amount of menatetrenone administered in a consolidation phase, wherein the consolidation phase is before the maintenance phase but after the initial induction phase.

In another embodiment, the amount of menatetrenone administered in a consolidation phase, is more than the amount of menatetrenone administered in the maintenance phase but less than the amount of menatetrenone administered in the initial induction phase.

In certain illustrative embodiments, the amount of menatetrenone administered in the maintenance phase is less than the amount of menatetrenone administered in the initial induction phase.

In certain illustrative embodiments, the initial induction phase period is about 30 to 120 days.

In certain illustrative embodiments, the initial induction phase period is about 60 to 90 days.

In certain illustrative embodiments, the maintenance phase period is at least 60 days.

In certain illustrative embodiments, the maintenance phase period is at least 90 days.

In certain illustrative embodiments, the maintenance phase period is at least 120 days.

In certain illustrative embodiments, the consolidation phase period is at least 60 days.

In certain illustrative embodiments, the consolidation phase period is at least 120 days.

In certain illustrative embodiments, the consolidation phase period is at least 180 days.

In certain illustrative embodiments, the menatetrenone is administered intravenously, orally, or subcutaneously.

In certain embodiments, the invention further comprises administering an angiotensin converting enzyme inhibitor.

In certain illustrative embodiments, the angiotensin converting enzyme inhibitor is enalapril, ramipril, quinapril, perindopril, lisinopril, benazepril, imidapril, or zofenopril.

In certain illustrative embodiments, the angiotensin converting enzyme inhibitor is ramipril.

In another embodiment, the invention encompasses methods of treating hepatocellular adenocarcinoma comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

In certain illustrative embodiments, the invention further comprises administering amount of menatetrenone administered in a consolidation phase, wherein the consolidation phase is before the maintenance phase but after the initial induction phase.

In another embodiment, the amount of menatetrenone administered in a consolidation phase, is more than the amount of menatetrenone administered in the maintenance phase but less than the amount of menatetrenone administered in the initial induction phase.

In certain illustrative embodiments, the invention further comprises administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

In certain illustrative embodiments, the amount of menatetrenone administered in the maintenance phase is less than the amount of menatetrenone administered in the initial induction phase.

In certain illustrative embodiments, the initial induction phase period is about 30 to 120 days.

In certain illustrative embodiments, the initial induction phase period is about 60 to 90 days.

In certain illustrative embodiments, the maintenance phase period is at least 60 days.

In certain illustrative embodiments, the maintenance phase period is at least 90 days.

In certain illustrative embodiments, the maintenance phase period is at least 120 days.

In certain illustrative embodiments, the consolidation phase period is at least 60 days.

In certain illustrative embodiments, the consolidation phase period is at least 120 days.

In certain illustrative embodiments, the consolidation phase period is at least 180 days.

In certain illustrative embodiments, the menatetrenone is administered intravenously, orally, or subcutaneously.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In another embodiment, the invention encompasses a method of treating hepatocellular adenocarcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 90 mg.

In another embodiment, the invention encompasses a method of treating hepatocellular adenocarcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 75 mg.

In another embodiment, the invention encompasses a method of treating hepatocellular adenocarcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 60 mg.

In another embodiment, the invention encompasses a method of treating hepatocellular adenocarcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 45 mg.

In another embodiment, the invention encompasses a method of treating hepatocellular adenocarcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 30 mg.

In another embodiment, the invention encompasses a method of treating hepatocellular adenocarcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 15 mg.

In another embodiment, the method further comprises administering menatetrenone in a consolidation phase after the initial induction phase but before the maintenance phase, wherein the menatetrenone is administered in a daily amount of about 250 mg.

In another embodiment, the method further comprises administering menatetrenone in a consolidation phase after the initial induction phase but before the maintenance phase, wherein the menatetrenone is administered in a daily amount of about 150 mg.

In another embodiment, the method further comprises administering menatetrenone in a consolidation phase after the initial induction phase but before the maintenance phase, wherein the menatetrenone is administered in a daily amount of about 100 mg.

In another embodiment, the method further comprises administering menatetrenone in a consolidation phase after the initial induction phase but before the maintenance phase, wherein the menatetrenone is administered in a daily amount of about 45 mg.

In another embodiment, the method further comprises administering menatetrenone in a consolidation phase after the initial induction phase but before the maintenance phase, wherein the menatetrenone is administered in a daily amount of about 30 mg.

In another embodiment, the method further comprises administering menatetrenone in a consolidation phase after the initial induction phase but before the maintenance phase, wherein the menatetrenone is administered in a daily amount of about 15 mg.

In another embodiment, the invention encompasses a method of treating hepatocellular adenocarcinoma comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 90 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In another embodiment, the invention encompasses a method of treating hepatocellular adenocarcinoma comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 45 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2, years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously.

In another embodiment, the invention encompasses a method of treating hepatocellular adenocarcinoma comprising administering to a subject in need thereof a therapeutically effective amount of menatetrenone according to the following dosing regimen:

(i) administering menatetrenone in an initial induction phase, wherein the menatetrenone is administered in a daily amount to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In another embodiment, the invention encompasses a method of treating hepatocellular adenocarcinoma comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously.

In another embodiment of the invention, the invention encompasses administering menatetrenone for about 180 consecutive days, optionally followed by a drug-free period of 2 to 30 consecutive days.

### IV. DETAILED DESCRIPTION OF THE INVENTION

The invention encompasses methods of treating hepatocellular adenocarcinoma comprising administering to a subject in need thereof a therapeutically effective amount of menatetrenone according to extended dose regimens. In accordance with the invention, a subject is administered an extended dose regimen of menatetrenone for a period of greater than 90 consecutive days.

An “extended dose regimen” of the invention refers to a regimen disclosed herein in which menatetrenone is administered for a period of greater than 90 consecutive days, wherein the menatetrenone is administered in at least two phases, an induction phase and a maintenance phase and optionally a consolidation phase, wherein a daily dosage of menatetrenone in a first phase is equal to or higher than a daily dosage of menatetrenone in a second phase, wherein a daily dosage of menatetrenone in a third phase is equal to or lower than the daily dosage of menatetrenone in the second phase, wherein a total daily dosage of menatetrenone in the second phase is equal to or lower than a total daily dosage of menatetrenone in the first phase, and wherein a total daily dosage of menatetrenone in the third phase is equal to or lower than the total daily dosage of menatetrenone in the second phase.

An “extended dose regimen” of the invention also refers to a regimen disclosed herein in which menatetrenone is administered for a period of greater than 90 consecutive days, wherein the menatetrenone is administered in at least two phases, wherein a total daily dosage of menatetrenone in a second phase is equal to or lower than a total daily dosage of estrogen and progestin in a first phase, and wherein a daily dosage of menatetrenone in the third phase is equal to or lower than the total daily dosage of menatetrenone in the second phase.

As used herein, “extended cycle regimen” refers to a regimen in which a menatetrenone composition is administered for a period of greater than 90 days.

As used herein, “subject” refers to any animal classified as a mammal, including humans and non-humans, such as, but not limited to, domestic and farm animals, zoo animals, sports animals, and pets.

The terms “treat” and “treatment” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a clinically significant response, without excessive levels of side effects. Treatment also includes prolonging survival as compared to expected survival if not receiving treatment.

The term “continuous” or “consecutive” in reference to “administration” means that the frequency of administration is at least once daily. Note, however, that the frequency of administration can be greater than once daily and still be “continuous,” e.g., twice or even three times daily, as long as the dosage levels as specified herein are not exceeded.

The term “daily dosage,” “daily dosage level,” “daily dosage amount,” or “daily dose” means the total amount of menatetrenone administered per day. Thus, for example, “continuous administration” of a menatetrenone to a subject at a “daily dosage level” of 90 mg means that the subject receives a total of 90 mg of menatetrenone on a daily basis, whether the menatetrenone is administered as a single 90 mg dose or, e.g., three separate 30 mg doses. A conventional means of continuously administering an menatetrenone is as a single daily oral dose at the prescribed daily dosage level.

### A. Dosages and Regimens

The invention encompasses a method of treating a subject with hepatocellular adenocarcinoma of providing an extended dosing regimen of menatetrenone, the method comprising administering to a subject in need thereof menatetrenone for a period of greater than 30 consecutive days, wherein the menatetrenone is administered in at least three phases, wherein a daily dosage of menatetrenone in a second phase is equal to or lower than a daily dosage of menatetrenone in a first phase, wherein a daily dosage of menatetrenone in a third phase is equal to or lower than the daily dosage of menatetrenone in the second phase.

The invention is also directed to a method of providing an extended dosing regimen, the method comprising administering to a subject in need thereof menatetrenone for a period of greater than 30 consecutive days, wherein the menatetrenone administered in at least two phases (e.g., induction and maintenance), wherein a total daily dosage of menatetrenone in a second phase is equal to or lower than a total daily dosage of menatetrenone in a first phase, and wherein a daily dosage of menatetrenone in the third phase is equal to or lower than the daily dosage of menatetrenone in the second phase.

In some embodiments of the invention, the daily dosage of menatetrenone in the first and second phases is equal to each other. In further embodiments of the invention, the daily dosages of menatetrenone in the first, second, and third phases are equal to each other. The daily dosage of menatetrenone can be, but is not limited to, the equivalent of 90 mg of menatetrenone daily for the first, second, and third phases.

In some embodiments of the invention, the daily dosage of menatetrenone in the second phase is lower than the daily dosage of menatetrenone in the first phase. In further embodiments of the invention, the daily dosage of menatetrenone in the third phase is lower than the daily dosage of menatetrenone in the second phase.

In some embodiments of the invention, the daily dosage of menatetrenone in the second phase is equal to the daily dosage of menatetrenone in the first phase and the daily dosage of menatetrenone in the third phase is lower than the daily dosage of menatetrenone in the second phase. In other embodiments of the invention, the daily dosage of menatetrenone in the second phase is lower than the daily dosage of menatetrenone in the first phase and the daily dosage of menatetrenone in the third phase is lower than the daily dosage of menatetrenone in the second phase.

The daily dosage of menatetrenone in the first phase can be, but is not limited to, about 45 mg to about 1000 mg, about 60 mg to about 500 mg, or about 90 mg to about 150 mg of menatetrenone. In certain embodiment, the daily dosage in any phase is about 15 mg, 30 mg, 45 mg, 60 mg, 75 mg, 90 mg, 105 mg, 120 mg, 135 mg, 150 mg, 225 mg, 450 mg, 600 mg, 800 mg, or 1000 mg. For example, the daily dosage of menatetrenone in the first initial phase can be 90 mg to 150 mg. The daily dosage of menatetrenone in the second phase can be, but is not limited to, about 30 mg to about 250 mg, about 60 mg to about 150 mg, or about 90 mg to about 135 mg of menatetrenone. For example, the daily dosage of menatetrenone in the second phase can be about 60 mg. The daily dosage of menatetrenone in the third phase can be, but is not limited to, about 15 mg to 125 mg, about 30 mg to about 90 mg, or about 45 mg to about 60 mg. For example, the daily dosage of menatetrenone in the third phase can be the equivalent of 45 mg.

In some embodiments of the invention, the method of providing an extended cycle regimen further includes a drug-free period. The drug-free period can be, but is not limited to, 2 to 10 consecutive days. The drug-free period can be 2 to 8 consecutive days. For example, the drug-free period can be 3, 5 or 7 days. The drug-free period can be a non-administration or administration of a placebo. The drug-free period can include administration of other active ingredients.

Examples of other additional pharmaceutically active ingredients or agents include, but are not limited to, vitamin D or vitamin D analogues; one or more of the B complex vitamins, such as vitamin B3 (niacin (i.e., nicotinic acid and/or nicotinamide)), vitamin B9 (folic acid or folate), vitamin B6 and/or vitamin B12; minerals such as, for example, calcium; iron (e.g., ferrous iron, such as, e.g., ferrous sulfate, ferrous fumarate, ferrous gluconate, or an iron glycine amino acid chelate).

These additional active agents can be administered during the period of administration of menatetrenone or the drug-free period. For example, vitamin D and/or calcium can be administered during the drug-free period. The active ingredients can be provided in the same, different, or separate dosage forms.

In some embodiments of the invention, the administration of an extended dose regimen of the invention is followed by monophasic administration of an menatetrenone. As used herein, “monophasic” refers to the continuous use of one particular dose of menatetrenone during the period of administration of the dosage form of the menatetrenone. In some aspects of the invention, the administration of an extended dose regimen is followed by monophasic administration of an menatetrenone continuously. In some aspects of the invention, the administration of an extended dose regimen is followed by monophasic administration of an menatetrenone for a period of greater than 30 or 31 consecutive days.

In some embodiments of the invention, the administration of an extended dose regimen is followed by monophasic administration of an menatetrenone for a period of about 350 to about 370 consecutive days, of about 260 to about 280 consecutive days, of about 175 to about 190 consecutive days, or of about 60 to about 110 consecutive days. The monophasic administration of menatetrenone can be optionally followed either by a drug-free period of, e.g., 2 to 10 consecutive days or by administration of menatetrenone for a period of, e.g., 2 to 10 consecutive days.

### B. Methods of Treatment

The invention generally encompasses methods of treating hepatocellular carcinoma comprising administering a therapeutically effective amount of menatetrenone to a subject in need thereof.

In certain embodiments, the invention encompasses suppression of cyclin D1 comprising administering menatetrenone to a subject according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the invention encompasses suppression of inflammatory cytokine nuclear factor κB (NF-κB) expression comprising administering menatetrenone to a subject according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the invention encompasses inhibition of matrix metalloproteinase (MMP) as a multikinase inhibitor expression comprising administering menatetrenone to a subject according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the invention encompasses suppressing the steroid and xenobiotic receptor (SXR)/pregnane X receptor (PXR) to modulate gene transcription in 1-ICC cells comprising administering menatetrenone to a subject according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the invention encompasses inhibiting cyclin D1 mRNA expression to modulate gene transcription in 1-ICC cells comprising administering menatetrenone to a subject according to the following dosing regimen:

(i) administering menatetrenone in an initial induction phase for a period of at least eight weeks, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In another embodiment, the invention encompasses a method of treating hepatocellular carcinoma comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 150 mg or greater than 150 mg.

In another embodiment, the invention encompasses a method of treating hepatocellular carcinoma comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 125 mg or greater than 125 mg.

In another embodiment, the invention encompasses a method of treating hepatocellular carcinoma comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 100 mg or greater than 100 mg.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In another embodiment, the invention encompasses a method of treating hepatocellular carcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 90 mg.

In another embodiment, the invention encompasses a method of treating hepatocellular carcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 75 mg.

In another embodiment, the invention encompasses a method of treating hepatocellular carcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 60 mg.

In another embodiment, the invention encompasses a method of treating hepatocellular carcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 45 mg.

In another embodiment, the invention encompasses a method of treating hepatocellular carcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 30 mg.

In another embodiment, the invention encompasses a method of treating hepatocellular carcinoma comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 15 mg.

In another embodiment, the method further comprises administering menatetrenone in a consolidation phase after the initial induction phase but before the maintenance phase, wherein the menatetrenone is administered in a daily amount of about 250 mg.

In another embodiment, the method further comprises administering menatetrenone in a consolidation phase after the initial induction phase but before the maintenance phase, wherein the menatetrenone is administered in a daily amount of about 150 mg.

In another embodiment, the method further comprises administering menatetrenone in a consolidation phase after the initial induction phase but before the maintenance phase, wherein the menatetrenone is administered in a daily amount of about 100 mg.

In another embodiment, the method further comprises administering menatetrenone in a consolidation phase after the initial induction phase but before the maintenance phase, wherein the menatetrenone is administered in a daily amount of about 45 mg.

In another embodiment, the method further comprises administering menatetrenone in a consolidation phase after the initial induction phase but before the maintenance phase, wherein the menatetrenone is administered in a daily amount of about 30 mg.

In another embodiment, the method further comprises administering menatetrenone in a consolidation phase after the initial induction phase but before the maintenance phase, wherein the menatetrenone is administered in a daily amount of about 15 mg.

In another embodiment, the invention encompasses a method of treating hepatocellular carcinoma comprising administering to a subject in need thereof a therapeutically effective amount of menatetrenone according to the following dosing regimen:

(1) administering menatetrenone in an initial induction phase for a period of at least 8 weeks, wherein the menatetrenone is administered in a daily amount of about 90 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In another embodiment, the invention encompasses a method of treating hepatocellular carcinoma comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 45 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously.

In another embodiment, the invention encompasses a method of treating hepatocellular carcinoma comprising administering to a subject in need thereof a therapeutically effective amount of menatetrenone according to the following dosing regimen:

(i) administering menatetrenone in an initial induction phase, wherein the menatetrenone is administered in a daily amount to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In another embodiment, the invention encompasses a method of treating hepatocellular carcinoma comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously

In another embodiment of the invention, the invention encompasses administering menatetrenone for about 180 consecutive days, optionally followed by a drug-free period of 2 to 30 consecutive days.

In certain embodiments, the menatetrenone is administered according to the following dosing regimen:

(i) administering menatetrenone in an initial induction phase for a period of at least 7 days, wherein the menatetrenone is administered in a daily amount of about 100 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of inducing apoptosis and differentiation in leukemic cells comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of treating hepatocellular carcinoma in a subject refractive to traditional chemotherapy (e.g., a subject not able to tolerate chemotherapeutic drugs, for example, lenalidomide or azacitidine) comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of treating hepatocellular carcinoma in a subject over 65 years old who cannot tolerate intensive myeloablative chemotherapy and/or stem cell transplantation comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of improving hematopoeisis comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

### C. Modes of Administration and Compositions

The menatetrenone is administered in the conventional manner by any route where they it is active. For example, administration can be by, but is not limited to, oral, parenteral, subcutaneous, intravenous, intramuscular, intraperitoneal, transdermal, buccal, or ocular routes, or by inhalation, by depot injections, or by implants. Thus, the dosage forms for the menatetrenone can be, but are not limited to, sublingual, injectable (including short-acting, depot, implant and pellet forms injected subcutaneously or intramuscularly), rectal suppositories, intrauterine devices, and transdermal forms such as patches and creams.

Thus, pharmaceutical compositions containing menatetrenone and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder. It is known in the art that the active ingredients can be contained in such compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, “Modern Pharmaceutics”, Banker & Rhodes, Marcel Dekker, Inc. 1979; and “Goodman & Gilman's The Pharmaceutical Basis of Therapeutics,” 6.sup.th Edition, MacMillan Publishing Co., New York 1980 can be consulted.

For oral administration, the menatetrenone can be formulated by combining with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

The pharmaceutical compositions of menatetrenone also can comprise suitable solid or gel phase carriers or excipients such as calcium carbonate, calcium phosphate, various sugars, starches, cellulose derivatives, gelatin, and polymers such as, e.g., polyethylene glycols.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All compositions for oral administration should be in dosages suitable for such administration.

For buccal administration, the menatetrenone compositions can take the form of tablets or lozenges formulated in conventional manner.

For administration by inhalation, the menatetrenone for use according to the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The menatetrenone can be formulated for parenteral administration by injection, e.g., by bolus injection or continuous infusion. The menatetrenone can be administered by continuous infusion subcutaneously over a period of about 15 minutes to about 24 hours. Compositions for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The menatetrenone can also be formulated in rectal compositions such as suppositories or retention enemas, e.g., containing conventional suppository bases such as cocoa butter or other glycerides.

In addition to the compositions described previously, the menatetrenone can also be formulated as a depot preparation. Such long acting compositions can be administered by implantation (e.g., subcutaneously or intramuscularly) or by intramuscular injection. Depot injections can be administered at about 1 to about 6 months or longer intervals. Thus, for example, the menatetrenone can be formulated with suitable polymeric or hydrophobic materials (e.g., as an emulsion in an acceptable oil) or ion exchange resins, or as sparingly soluble derivatives, for example, as a sparingly soluble salt.

For transdermal administration, the menatetrenone can be applied by any transdermal, therapeutic system that is consequently supplied to the organism, such as, for example, as a transdermal patch, transdermal cream or plaster. For example, the menatetrenone can be formulated as a transdermal patch. The preparation and use of transdermal patches are well known to those of skill in the art and are available in different designs, including matrix-type or reservoir-type designs. In addition to the estrogen and/or progestin, transdermal patches can contain additional components such as penetration-enhancing agents and/or additional excipients that are conventionally employed, such as, e.g., carriers, gelling agents, suspending agents, dispersing agents, preservatives, stabilizers, wetting agents, emulsifying agents, and the like.

The menatetrenone can also be administered with other active ingredients. The drug-free period or the unopposed estrogen interval can also include administration of other active ingredients. For example, as described above, menatetrenone can also be administered with vitamin D and/or calcium in the extended cycle regimens. Alternatively, vitamin D and/or calcium can be administered in the extended cycle regimens during the unopposed menatetrenone interval following administration of menatetrenone. The form of vitamin D and of calcium used in the invention would be well known to those of skill in the art, as would the amount. For example, calcium can be administered in the form of calcium carbonate, at a dosage level of e.g., 500 mg.

In some embodiments of the invention, the menatetrenone is in a oral, transdermal, or injectable liquid dosage form. For example, the menatetrenone can be in an oral dosage form or a transdermal dosage form.

In some embodiments of the invention, each phase of the regimen of the invention can be administered in a separate, single dosage form. In other aspects of the invention, each phase of the regimen of the invention can be administered in one or more separate dosage forms. For example, each phase can be administered using a transdermal device (such as a patch).

### D. Kits

The dosages or compositions for the extended cycle regimens of the invention can be provided in the form of a kit or package, with the dosages arranged for proper sequential administration. For example, in the oral form of the composition, the invention provides a pharmaceutical package, which contains multiple dosage units in a synchronized, fixed sequence, wherein the sequence or arrangement of the dosage units corresponds to the stages of daily administration.

Thus, for example, the pharmaceutical compositions useful in the invention can be provided in kit form containing greater than 30 or 31 tablets intended for ingestion on successive days. In some embodiments, the kit further contains, e.g., 2 to 10 tablets, intended for ingestion on successive days following the ingestion of the greater than 30 or 31 tablets. Administration is daily for a period of greater than 30 or 31 consecutive days using tablets containing menatetrenone, and, in some embodiments, is followed by administration that is daily for, e.g., 2 to 10 consecutive days using either placebo tablets or tablets containing no menatetrenone. For example, administration can be for 40-190 consecutive days, using tablets containing menatetrenone, followed by administration for, e.g., at least 2-10 days using tablets containing no menatetrenone. As another example, administration can be for 75-95 days, using tablets containing menatetrenone, followed by administration for, e.g., at least 2-10 days using tablets containing no menatetrenone. As yet another example, administration can be for 168-186 days, using tablets containing menatetrenone, followed by administration for, e.g., at least 2-10 days using tablets containing no menatetrenone.

Some embodiments of the invention provide a pharmaceutical kit comprising a first transdermal device capable of providing a daily dosage of menatetrenone; a second transdermal device capable of providing a daily dosage of menatetrenone that is equal to or higher than that of the first transdermal device and capable of providing a total daily dosage of menatetrenone that is higher than that of the first transdermal device; and a third transdermal device capable of providing a daily dosage of menatetrenone that is equal to or higher than that of the second transdermal device and capable of providing a total daily dosage of estrogen and progestin that is higher than that of the second transdermal device; wherein the transdermal devices are capable of providing menatetrenone for a period of greater than 30 or 31 consecutive days.

Some embodiments of the invention provide a pharmaceutical kit comprising a first oral dosage capable of providing a daily dosage of menatetrenone; a second oral dosage capable of providing a daily dosage of menatetrenone that is equal to or lower than that of the first oral dosage and capable of providing a total daily dosage of menatetrenone that is higher than that of the first oral dosage; and a third oral dosage capable of providing a daily dosage of menatetrenone that is equal to or lower than that of the second oral dosage and capable of providing a total daily dosage of menatetrenone that is higher than that of the second oral dosage; wherein the oral dosages are capable of providing menatetrenone for a period of greater than 30 or 31 consecutive days.

The pharmaceutical kits of the invention can further include instructions for proper sequential administration in accordance with the regimens of the invention.

The invention also encompasses a method of delivering a pharmaceutical composition for an extended dose regimen of the invention to a patient in need thereof, the method comprising (a) registering in a computer readable medium the identity of a physician permitted to prescribe a pharmaceutical composition for an ascending-dose extended cycle regimen; (b) providing the patient with counseling information concerning the risks attendant to the pharmaceutical composition; (c) obtaining informed consent from the patient to receive the pharmaceutical composition despite the attendant risks; (d) registering the patient in a computer readable medium after obtaining their informed consent; and (e) permitting the patient access to the pharmaceutical composition.

The drug delivery methods of the invention involve, inter alia, registering in a computer readable storage medium physicians who are qualified to prescribe the ascending-dose extended cycle regimen of the present invention. Once registered in the computer readable storage medium, the physician can be eligible to prescribe the pharmaceutical composition to a patient in need thereof. Generally speaking, in order to become registered in the computer readable storage medium, the physician may be required to comply with various aspects of, for example, providing patient education and counseling. The registration of the physician in the computer readable storage medium can be achieved by providing the physician, for example, by mail, facsimile transmission, or on-line transmission, with a registration card or form, preferably together with educational materials concerning the pharmaceutical composition of the present invention. The physician can complete the registration card or form by providing information requested therein, and the registration card or form can be returned to the manufacturer or distributor of the pharmaceutical composition of the present invention, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, the physician may determine that the patient's condition can be improved by the administration of the pharmaceutical composition of the invention. Prior to prescribing the pharmaceutical composition of the invention, the physician can counsel the patient, for example, on the various risks and benefits associated with the pharmaceutical composition of the invention. The patient can be provided full disclosure of all the known and suspected risks associated with the pharmaceutical composition of the present invention. Such counseling can be provided verbally, as well as in written form. In some embodiments, the physician can provide the patient with literature materials on the pharmaceutical composition of the present invention, such as product information, educational materials, and the like.

In addition to receiving counseling on the risks attendant to the pharmaceutical composition of the present invention, the methods of the invention further require the patient to fill out an informed consent form which is signed by the patient. Upon the completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the pharmaceutical composition of the present invention. Thus, the computer readable storage medium can serve to deny access to patients who fail to abide by the methods of the present invention. In some embodiments, access to the pharmaceutical composition of the present invention is in the form of a prescription, wherein the prescribing physician is registered in a computer readable storage medium, has provided counseling to the patient concerning the attendant risks of the pharmaceutical composition of the present invention, and has obtained informed consent from the patient, prior to prescribing the pharmaceutical composition of the present invention to the patient in need thereof.

All of the various aspects, embodiments and options described herein can be combined in any and all variations. The following examples are illustrative, but not limiting, of the method and compositions of the present invention. Other suitable modifications and adaptations of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance with the following claims and their equivalents.

### V. EXAMPLES

### Prophetic Example 1

To determine the effects of MK4 on cell growth, MK4 is added to a medium of HCC cell lines. MK4 inhibits HepG2 cell growth in a dose-dependent manner. The cell growth rate for 5 days will be lower in the MK4—treated cells than with untreated cells. The cell growth rate after a 5-day incubation will be greater in control cells than in cells that are treated with MK4. MK4 concentration will be shown to cause cell growth inhibition.

### Prophetic Example 2

To investigate the mechanism of suppression of MMP-1, -3 and -7 expression by

MK4, HCC cells will be transiently transfected with MMP-1, -3 and -7 promoter/luciferase reporter plasmids and then treated with MK4. MK4 dose-dependently will reduce MMP-1 and -7 promoter activity and decrease MMP-3 promoter activity weakly in the HepG2 cells. Similar results should be obtained from the Huh7 and HLE cells.

### Prophetic Example 3

Since MK4 inhibits MMP-1, -3 and -7 mRNA expression, the ability of MK4 to suppress TPA-induced MMP expression will be investigated. MK4 dose-dependently inhibits MMP-1, -3 and -7 mRNA expression induced by TPA. MK4 also suppresses the MMP-1 and -3 protein expression induced by TPA. Furthermore, MK4 dose-dependently suppresses the MMP-1, -3 and -7 promoter activity induced by TPA, which suggests that MK4 might inhibit the MMP expression through a PKC-mediated pathway.

### Prophetic Example 4

A subject suffering from hepatocellular carcinoma is administered oral MK4 (90 mg daily once a day). After the administration of MK4 for six weeks, tumor sizes will decrease and the diameter of the main tumor is smaller. This demonstrates that administration of MK-4 will cause the tumor to regress and the margin of the tumor to become obscure. After six weeks, MK-4 is then administered for an additional twenty-two months (45 mg daily once a day), no signs of recurrence are observed and tumor markers remain within the normal limits. This case report provides compelling evidence that MK4 may be useful in the treatment of HCC.

### Prophetic Example 5

Treatment with MK4 and an ACE-I normalizes AFP and AFP-L3 tumor markers and the hepatic nodule disappears. A subject with hepatitis C virus (HCV)-related liver cirrhosis is diagnosed with what was termed a “dysplastic nodule or early HCC.” The diagnosis is based on enhanced computed tomography (CE-CT) study and elevations of the tumor markers alpha-fetoprotein (AFP) and lectin-reactive alpha-fetal protein (AFP-L3).

Oral administration of MK4 (90 mg daily once a day) and ACE-I (e.g., perindopril; 4 mg/day), will cause the serum levels of both AFP and AFP-L3 to gradually decrease. After one-year treatment, MK-4 is then administered for an additional 48 months (45 mg daily once a day) and ACE-1 administration is halted, the serum levels of AFP and AFP-L3 should remain in the normal range. After 15 months of treatment, the hepatic nodule will disappear in both the arterial and equilibrium phases of the CE-CT study and continued maintenance with 45 mg/day MK4 results in no increase in AFP or AFP-L3 levels. This combined treatment is continued, and no HCC should develop during follow up. In conclusion, a patient with HCV-related cirrhosis is administered an initial combined treatment of MK4 and an ACE-I and then maintenance with MK$ alone resolves elevated serum levels of AFP and AFP-L3 and eliminates dysplastic nodule.

### Prophetic Example 6

Patients with hepatocellular carcinoma are randomized to receive oral MK4 (90 mg/day) or no MK4. All patients receive conventional therapy with percutaneous tumor ablation and/or transcatheter arterial embolization. The rate of portal vein invasion at 12 months of follow-up will be reduced in MK4 recipients as compared with controls. After 12 months of received 90 mg/day MK4, MK-4 is then administered for an additional twenty-two months (45 mg daily once a day), no signs of recurrence are observed and tumor markers remain within the normal limits.

Application of the compounds, compositions and methods of the present invention for the medical or pharmaceutical uses described can be accomplished by any clinical, medical, and pharmaceutical methods and techniques as are presently or prospectively known to those skilled in the art. It will therefore be appreciated that the various embodiments which have been described above are intended to illustrate the invention and various changes and modifications can be made in the invention method without departing from the spirit or scope thereof.

Having now fully described this invention, it will be understood to those of ordinary skill in the art that the same can be performed within a wide and equivalent range of conditions, compositions, and other parameters without affecting the scope of the invention or any embodiments thereof. All patents, patent applications, and publications cited herein are fully incorporated by reference herein in their entirety.

What is claimed is:

1. A method of inhibiting vascular endothelial growth factor comprising administering menatetrenone to a subject according to the following dosing regimen:
(i) administering menatetrenone in an initial induction phase for a period of at least eight weeks, wherein the menatetrenone is administered in a daily amount of about 60 mg to about 500 mg; and
(ii) administering menatetrenone in a maintenance phase after said initial induction phase, wherein the menatetrenone is administered in a daily amount of about 15 mg to about 135 mg.

2. The method of claim 1, wherein the subject is suffering from hepatocellular adenocarcinoma.

3. The method of claim 1, wherein the amount of menatetrenone administered in the maintenance phase is less than the amount of menatetrenone administered in the initial induction phase.

4. The method of claim 1, wherein the initial induction phase period is about 30 to 120 days.

5. The method of claim 1, wherein the initial induction phase period is about 60 to 90 days.

6. The method of claim 1, wherein the maintenance phase period is at least 60 days.

7. The method of claim 1, wherein the maintenance phase period is at least 90 days.

8. The method of claim 1, wherein the maintenance phase period is at least 120 days.

9. The method of claim 1, wherein the menatetrenone is administered intravenously, orally, or subcutaneously.

10. The method of claim 1, further comprising administering an angiotensin converting enzyme inhibitor.

11. The method of claim 1, wherein the angiotensin converting enzyme inhibitor is enalapril, ramipril, quinapril, perindopril, lisinopril, benazepril, imidapril, or zofenopril.

12. The method of claim 1, wherein the angiotensin converting enzyme inhibitor is ramipril.

13. A method of treating hepatocellular adenocarcinoma comprising administering to a subject in need thereof a therapeutically effective amount of menatetrenone according to the following dosing regimen:
(i) administering menatetrenone in an initial induction phase for a period of at least eight weeks, wherein the menatetrenone is administered in a daily amount of about 60 mg to about 500 mg; and
(ii) administering menatetrenone in a maintenance phase after said initial induction phase, wherein the menatetrenone is administered in a daily amount of about 15 mg to about 135 mg.

14. The method of claim 13, wherein the amount of menatetrenone administered in the maintenance phase is less than the amount of menatetrenone administered in the initial induction phase.

15. The method of claim 13, wherein the initial induction phase period is about 30 to 120 days.

16. The method of claim 13, wherein the initial induction phase period is about 60 to 90 days.

17. The method of claim 13, wherein the maintenance phase period is at least 60 days.

18. The method of claim 13, wherein the maintenance phase period is at least 90 days.

19. The method of claim 13, wherein the maintenance phase period is at least 120 days.

20. The method of claim 14, wherein the consolidation phase period is at least 60 days.

21. The method of claim 14, wherein the consolidation phase period is at least 120 days.

22. The method of claim 15, wherein the consolidation phase period is at least 180 days.

23. The method of claim 13, wherein the menatetrenone is administered intravenously, orally, or subcutaneously.

24. The method of claim 13, further comprising administering an angiotensin converting enzyme inhibitor.

25. The method of claim 13, wherein the angiotensin converting enzyme inhibitor is enalapril, ramipril, quinapril, perindopril, lisinopril, benazepril, imidapril, or zofenopril.

26. The method of claim 13, wherein the angiotensin converting enzyme inhibitor is ramipril.

27. A method of treating hepatocellular adenocarcinoma comprising administering to a subject in need thereof a therapeutically effective amount of menatetrenone according to the following dosing regimen:
(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and
(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

28. The method of claim 27, further comprising administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

29. The method of claim 27, wherein the amount of menatetrenone administered in the maintenance phase is less than the amount of menatetrenone administered in the initial induction phase.

30. The method of claim 27, wherein the initial induction phase period is about 30 to 120 days.

31. The method of claim 27, wherein the initial induction phase period is about 60 to 90 days.

32. The method of claim 27, wherein the maintenance phase period is at least 60 days.

33. The method of claim 27, wherein the maintenance phase period is at least 90 days.

34. The method of claim 27, wherein the maintenance phase period is at least 120 days.

35. The method of claim 28, wherein the consolidation phase period is at least 60 days.

36. The method of claim 28, wherein the consolidation phase period is at least 120 days.

37. The method of claim 28, wherein the consolidation phase period is at least 180 days.

38. The method of claim 27, wherein the menatetrenone is administered intravenously, orally, or subcutaneously.
